SEQUENCE LISTING (18 sequences)

SEQ ID NO: 1 - protein, 7 aa, synthetic construct
ENLYFQS

SEQ ID NO: 2 - protein, 5 aa, synthetic construct
LRGAG

SEQ ID NO: 3 - protein, 5 aa, synthetic construct
EEGEG

SEQ ID NO: 4 - protein, 10 aa, synthetic construct
LEEGEGLARL

SEQ ID NO: 5 - protein, 5 aa, synthetic construct
LRGAG

SEQ ID NO: 6 - protein, 5 aa, synthetic construct
EEGEG

SEQ ID NO: 7 - protein, 8 aa, synthetic construct
LEVLFQGP

SEQ ID NO: 8 - protein, 8 aa, synthetic construct
RAGGYIFS

SEQ ID NO: 9 - protein, 15 aa, synthetic construct
DELRLDRAGGYIFSS

SEQ ID NO: 10 - protein, 8 aa, synthetic construct
RAGAGIIE

SEQ ID NO: 11 - protein, 15 aa, synthetic construct
VEQLEDRAGAGIIET

SEQ ID NO: 12 - protein, 8 aa, synthetic construct
ERKRRGAD

SEQ ID NO: 13 - protein, 8 aa, synthetic construct
AAGKRGAA

SEQ ID NO: 14 - protein, 8 aa, synthetic construct
LVKRRGGG

SEQ ID NO: 15 - protein, 8 aa, synthetic construct
SAVLQSGF

SEQ ID NO: 16 - protein, 8 aa, synthetic construct
VARLQSGF

SEQ ID NO: 17 - protein, 8 aa, synthetic construct
VVRLQSGF

SEQ ID NO: 18 - protein, 322 aa, Escherichia phage P1
SDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLED

(19) United States
(12) Patent Application Publication
(10) Pub. No.: US 2026/0002137 A1
(43) Pub. Date: Jan. 1, 2026

(54) Protease-conditional targeted nucleic acid recombination, method and uses thereof

(71) Applicant: Instituto de Biologia Experimental e Tecnológica – iBET, Oeiras (PT)

(72) Inventors: Ana Sofia DE SOUSA VALENTE COROADINHA, LISBOA (PT); Miguel Ricardo GUERREIRO, LISBOA (PT); Ana Isabel MATOS DE ALMEIDA, SÃO DOMINGOS DE RANA (PT)

(73) Assignee: Instituto de Biologia Experimental e Tecnológica – iBET, Oeiras (PT)

(21) Appl. No.: 19/114,714

(22) PCT Filed: Sep. 21, 2023
(86) PCT No.: PCT/IB2023/059356

§ 371 Date: Mar. 24, 2025

(30) Foreign Application Priority Data
Sep. 22, 2022 (PT) 118205

(51) Int. Cl. C12N 9/12; C12Q 1/48

(52) U.S. Cl. CPC C12N 9/1241; C12Q 1/485; C07K 2319/50; C07K 2319/60; C12Y 207/07

(57) ABSTRACT

The present disclosure relates to enzymes, compositions and methods for performing conditional homologous recombination of a targeted DNA molecule or genome by using modified proteins comprising nucleic acid DNA binding proteins with protease-conditional recombinase (hereinafter “ProRec”) activity. Namely a new cre recombinase pro enzyme, compositions/kit and sensors comprising the cre recombinase pro enzyme of the present disclosure. Namely, a cre recombinase pro-enzyme, for identifying and quantifying proteolytic activity in a disease or an infection, comprising intein mediated circularization, a flip-excision cassette comprising an output reporter and/or effector protein; a linker sequence cleavable by a protease.

### CROSS-REFERENCE TO RELATED APPLICATIONS

This application is a U.S. National Stage Application under 35 U.S.C. § 371 of International Patent Application No. PCT/IB2023/059356, filed Sep. 21, 2023, which claims priority to Portugal Patent Application No. 118205, filed Sep. 22, 2022, the contents of which are hereby incorporated by reference in their respective entireties.

### SEQUENCE LISTING

This instant application contains a Sequence Listing which has been submitted electronically in ASCII format and is hereby incorporated by reference in its entirety. Said ASCII copy, created on Dec. 13, 2023, is named 10224_013084-USO_ST26.xml and is 17,067 bytes in size.

### TECHNICAL FIELD

The present disclosure relates to enzymes, compositions and methods for performing conditional homologous recombination of a targeted DNA molecule or genome by using modified proteins comprising nucleic acid DNA binding proteins with protease-conditional recombinase (hereinafter “ProRec”) activity. Namely a new cre recombinase pro enzyme, compositions/kit and sensors comprising the cre recombinase pro enzyme of the present disclosure.

### BACKGROUND

The simplicity of the Cre/lox and other recombinase systems revolutionized the gene editing toolbox, allowing new and innovative strategies of DNA genetic engineering. However, the always-on nature and the lack of effective activity control limits the applicability of recombinase based genetic circuits, and on sensing and recording cellular events. The Cre recombinase is a DNA-binding recombinase enzyme that has been adapted to enable the modification or perturbation of genes as well as regulation of non-coding genomic elements in a wide variety of organisms. There have been multiple attempts to create Cre recombinase variants with different purposes by fusing protein domains directly to the N- or C-terminus of Cre recombinase. However, a strategy to conditionally, but reversibly, eliminate protein activity, has not been developed. This strategy would allow the engineering of a regulated Cre permitting its conditional control of activity and allow precise examination of development, disease progression and cell differentiation depending on the existence of an effector molecule. The effector molecule herein described are classified as protease enzymes. Proteases can be found in all forms of life and viruses and are involved in many biological functions, including cell signaling and disease progression.

There is a need to profile the inherent plasticity of the Cre recombinase structure by examining its ability to tolerate structural distortion inactivation while retaining the ability to regain its activity through the incorporation of synthetic cleavable linkers.

Cre recombinase (hereinafter “Cre”) is a DNA-binding small bacteriophage P1-derived tyrosine recombinase enzyme. It uses a topoisomerase I-like mechanism to catalyze DNA recombination events between two specific DNA recognition sites, named loxP sites (locus of crossing over in phage P1), without requiring high-energy cofactors. The 34 base pairs (herein after “bp”) loxP sites are composed by two 13 bp palindromic regions (recombinase binding elements) flanking an asymmetric 8 bp spacer sequence. The latter determines orientation of the loxP sites. When a DNA fragment is flanked by two lox sites in the same orientation, Cre excises it as a circular molecule, leaving a single lox site behind. Conversely, when lox sites are in opposing orientations, Cre reverses the flanked DNA. Moreover, the spacer sequence also defines the identify of a target site, as heterospecific mutants efficiently cross-interact with one another, but rarely with the wild type lox site. The simplicity of the Cre system has led to its widespread usage in innovative strategies of DNA recombination, from fundamental studies on reversible immortalization of cells and organization of the nervous system, to biotechnological processes such as development of retroviral and adenoviral producer cell lines. Taking particular advantage of Cre/loxP systems are the fields on functional annotation of the human genome and modelling of human disease in mouse models, where complex recombinase-mediated cassette exchange (RMCE) approaches are commonly employed. Despite having revolutionized the gene editing toolbox, a number of issues still require particular attention. When expressed for a prolonged period of time or at a high level, the always-on nature of Cre leads to interaction of loxP-like sequences in the mammalian genome, which can result in chromosomal rearrangements and altered cell physiology. Several strategies circumventing these limitations have been developed, whether by regulation of the transcription of cre gene by inducible promoters, or by impairing its migration to the cell nucleus, for example by fusion of Cre to the ligand-binding domain of a steroid receptor. Other strategies have pursued to control transcomplementation of inactive Cre fragments, whether by attachment of fragments to interacting protein partners, coiled-coil domains, or split inteins. However, these systems are not tight enough to prevent Cre leaky expression or completely impair Cre functionality. Moreover, split Cre versions have the disadvantage of lower recombination efficiency when compared to the full-length version. Given its potential, the lack of activity control limits the applicability of Cre on genetic circuits sensing and recording cellular events.

Validating Cre recombinase as a powerful toolbox, a Cre-based sensor activated by dengue virus (DENV) proteolytic activity was recently reported. Therein, full-length Cre recombinase remains localized in the endoplasmic reticulum through fusion to the C-terminus of NS4B-_{N10}NS5, until its release by the viral protease. Cre recombinase translocation to the nucleus leads to removal of floxed stop codons and a polyadenylation signal upstream a GFP reporter. Cre recombinase system revolutionized the gene editing toolbox due to its simplicity and power, allowing new and innovative strategies of DNA recombination in fundamental, biotechnological, or even biomedical research. However, the lack of effective control of the always-on nature of the recombinase limits its applicability on genetic circuits sensing and recording cellular events.

These facts are disclosed in order to illustrate the technical problem addressed by the present disclosure.

### REFERENCES

(1) Abremski, K.; Hoess, R. Bacteriophage P1 site-specific recombination. Purification and properties of the Cre recombinase protein. J. Biol. Chem. 1984, 259, 1509-1514.
(2) Hoess, R.H.; Abremski, K. Mechanism of strand cleavage and exchange in the Cre-lox site-specific recombination system. J. Mol. Biol. 1985, 181, 351-362.
(3) Kobayashi, N.; Noguchi, H.; Westerman, K.A.; Matsumura, T.; Watanabe, T.; Totsugawa, T.; Fujiwara, T.; Leboulch, P.; Tanaka, N. Efficient Cre/loxP site-specific recombination in a HepG2 human liver cell line. Cell Transplant. 2000, 9, 737-742.
(4) Saunders, A.; Johnson, C.A.; Sabatini, B.L. Novel recombinant adeno-associated viruses for Cre activated and inactivated transgene expression in neurons. Front. Neural Circuits 2012, 6, 1-10.
(5) Coroadinha, A.S.; Schucht, R.; Gama-Norton, L.; Wirth, D.; Hauser, H.; Carrondo, M.J.T. The use of recombinase mediated cassette exchange in retroviral vector producer cell lines: Predictability and efficiency by transgene exchange. J. Biotechnol. 2006, 124, 457-468.
(6) Fernandes, P.; Santiago, V.M.; Rodrigues, A.F.; Tomás, H.; Kremer, E.J.; Alves, P.M.; Coroadinha, A.S. Impact of E1 and Cre on Adenovirus Vector Amplification: Developing MDCK CAV-2-E1 and E1-Cre Transcomplementing Cell Lines. PLOS One 2013, 8, e60342.
(7) Turan, S.; Zehe, C.; Kuehle, J.; Qiao, J.; Bode, J. Recombinase-mediated cassette exchange (RMCE)-A rapidly-expanding toolbox for targeted genomic modifications. Gene 2013, 515, 1-27.
(8) Loonstra, A.; Vooijs, M.; Beverloo, H.B.; Allak, B.A.; van Drunen, E.; Kanaar, R.; Berns, A.; Jonkers, J. Growth inhibition and DNA damage induced by Cre recombinase in mammalian cells. Proc. Natl. Acad. Sci. 2001, 98, 9209-9214.
(9) Mattheakis, L.C.; Olivan, S.E.; Dias, J.M.; Northrop, J.P. Expression of Cre recombinase as a reporter of signal transduction in mammalian cells. Chem. Biol. 1999, 6, 835-844.
(10) Kristianto, J.; Johnson, M.G.; Zastrow, R.K.; Radcliff, A.B.; Blank, R.D. Spontaneous recombinase activity of Cre-ERT2 in vivo. Transgenic Res. 2017, 26, 411-417.
(11) Hirrlinger, J.; Scheller, A.; Hirrlinger, P.G.; Kellert, B.; Tang, W.; Wehr, M.C.; Goebbels, S.; Reichenbach, A.; Sprengel, R.; Rossner, M.; et al. Split-Cre complementation indicates coincident activity of different genes in vivo. PLOS One 2009, 4, e4286.
(12) Schnütgen, F.; Doerflinger, N.; Calleja, C.; Wendling, O.; Chambon, P.; Ghyselinck, N.B. A directional strategy for monitoring Cre-mediated recombination at the cellular level in the mouse. Nat. Biotechnol. 2003, 21, 562-565.
(13) Kapust, R.B.; Tözsér, J.; Fox, J.D.; Anderson, D.E.; Cherry, S.; Copeland, T.D.; Waugh, D.S. Tobacco etch virus protease: mechanism of autolysis and rational design of stable mutants with wild-type catalytic proficiency. Protein Eng. Des. Sel. 2001, 14, 993-1000.
(14) Hsieh, M.S.; Chen, M.Y.; Hsieh, C.H.; Pan, C.H.; Yu, G.Y.; Chen, H.W. Detection and quantification of dengue virus using a novel biosensor system based on dengue NS3 protease activity. PLOS One 2017, 12, e0188170.

### GENERAL DESCRIPTION

The present disclosure relates to compositions and methods for performing conditional homologous recombination of a targeted DNA molecule or genome by using modified proteins comprising nucleic acid DNA binding proteins with protease-conditional recombinases (hereinafter “ProRec”) activity.

The present disclosure relates to a novel method of regulating the activity of highly functional Cre sensor modules using unique structural distortion schemes. An engineered Cre harboring a linker sequence is maintained in a non-functional pro-enzyme state by Nostoc punctiforme DnaE split intein (Npu DnaE) mediated protein cyclization or similar protein trans-splicing systems (e.g. consensus sequence from this alignment (Cfa), synthetic split inteins derived from gp41, etc). By using protease-specific cleavage sites as the linker sequence, Cre functionality can be efficiently restored upon proteolytic cleavage. A fluorescence reporter module dependent on Cre-mediated “flip-excision” (FLEx) ultimately transfers the potentially transient proteolytic activity into a permanent output signal. In an initial proof-of-concept, the unique design of this protease-sensing Cre recombinase (ProRec) module was characterized and validated in response to viral proteases, with the aim of establishing a cellular platform for detection and quantification of infectious label-free viruses and viral vectors.

The method presented herein, where the ProRec sensor has the potential to detect viral or cellular proteolytic activity, and the FLEx reporter can robustly output a permanent signal, provides a toolbox for new and innovative applications relying on tightly controlled and highly functional recombinase variants. It can also be used as regulatable synthetic circuit where the protease will be the inducer that activates expression of the FLEx regulatable cassette.

The present disclosure relates to compositions and methods for performing conditional homologous recombination of a targeted DNA molecule or genome for enabling, deleting, inverting or integrating nucleic acids. The deletion, inversion or integration of nucleic acids may result in the start or stop of gene transcription.

An aspect of the present disclosure relates to modified proteins comprising nucleic acid DNA binding proteins with protease-conditional recombinases activity, named ProRec. The ProRec protein of the present disclosure have been altered (relative to a standard Rec) to be in pro-enzyme state by fusing their C and N terminal with polypeptides capable of providing structural distortion and shut-off of the enzymatic activity. The ProRec protein of the present disclosure also contains a polypeptide sequence recognized by specific proteases enabling ProRec to become active. ProRec proteins herein described are based on Cre recombinase but other site specific recombinase enzymes could be used, such as flp, Phi or Dre recombinases.

An aspect of the present disclosure relates to FLEX (“flip-excision”) Cre-Switch system enabling conditional inducible protein expression, comprising of targeted DNA sequences, by ProRec proteins and protease.

Another aspect of the present disclosure relates to methods of using the ProRec protein of the present disclosure as a sensor of proteolytic activity (e.g. from viral and cellular proteases). The methods can be performed in vitro and in vivo. Namely it can be used in animal models to monitor infection status (active vs latency). It can also be used in advanced therapeutic medicinal products (ATMPs) (e.g. cell and gene therapies) to provide treatment.

The protein and method of the present disclosure presents two potential important advantages as a virus-sensing platform. ProRec does not remain localized on specific cellular structures. As such, it is expected that transient interactions between sensor and protease molecules occur with increased frequency and efficiency. Moreover, FLEx reporter module does not allow basal reporter expression before DNA recombination, contrasting with the “transcriptional stopper” element used for DENV detection which may not completely block read-through expression of the downstream fluorescent reporter.

Cre recombinase pro-enzyme, for identifing and quantifing proteolytic activity in a disease or an infection, comprising intein mediated circularization, a flip-excision cassette comprising an output efector and/or reporter protein, and a linker sequence cleavable by a protease for detecting proteases and quantifing proteolytic activity.

In an embodiment, the flip-excision cassette comprises a reporter protein.

In an embodiment, the flip-excision cassette comprises an output effector protein.

An aspect of the present dislosure relates to a Cre recombinase pro-enzyme, for identifing and quantifing proteolytic activity in disease or infection comprising intein mediated circularization, a flip-excision cassette comprising asignal protein, preferably an output signal protein and/or a protein; even more in particular a reporter, an effector protein, or both in a theranostic circuit; a linker sequence cleavable by a protease for detecting proteases and quantifing proteolytic activity.

Output Signal Protein (also known as Effector or Target Protein) are the downstream components of a signaling pathway that carry out the actual cellular responses or effects in response to a signaling event. These proteins can be enzymes, transcription factors, or other molecules that are activated or modified as a result of signaling events initiated by signal proteins. Output signal proteins mediate the cellular responses, such as changes in gene expression, enzyme activity, or cell behavior, in response to the initial signal. Output signal proteins can be located within the cell, where they can directly influence cellular processes.

A reporter protein is a protein that is genetically engineered or tagged to produce a detectable signal, typically a fluorescent, luminescent, or colorimetric signal, when it is expressed or activated within a cell or organism. Reporter proteins are used to track gene expression, protein localization, or cellular processes. They allow visualization and measurement of the activity of specific genes or proteins in living cells or organisms. Green Fluorescent Protein (GFP), Luciferase, β-Galactosidase, and other fluorescent or luminescent markers are commonly used as reporter proteins.

An aspect of the present dislosure relates to a Cre recombinase pro-enzyme, for identifing and quantifing proteolytic activity in a disease or an infection, comprising intein mediated circularization, a flip-excision cassette comprising a reporter protein, and a linker sequence cleavable by a protease for detecting proteases and quantifing proteolytic activity.

In an embodiment for better results, the intein mediated circularization is a N-terminal-C-terminal fused split intein mediated circularization.

In an embodiment for better results, the linker sequence cleavable comprises from 4-15 amino acids; preferably from 4-12 amino acids, more preferably 4-10 amino acids.

In an embodiment for better results, the linker sequence cleavable is selected from SEQ. ID. 1: ENLYFQ↓S, SEQ. ID. 2: LRGA↓G, SEQ. ID. 3: EEGE↓G, SEQ. ID. 4: LEEGE↓GLARL, SEQ. ID. 5: LRGA↓G, SEQ. ID. 6: EEGE↓G, SEQ. ID. 7: LEVLFQ↓GP; SEQ. ID. 8: RAGG↓YIFS,; SEQ. ID. 9: DELRLDRAGG↓YIFSS, SEQ. ID. 10: RAGA↓GIIE, SEQ. ID. 11: VEQLEDRAGA↓GIIET, SEQ. ID. 12: ERKRR↓GAD, SEQ. ID. 13: AAGKR↓GAA, SEQ. ID. 14: LVKRR↓GGG, SEQ. ID. 15: SAVLQ↓SGF, SEQ. ID. 16: VARLQ↓SGF, SEQ. ID. 17: VVRLQ↓SGF.

In an embodiment for better results, the split inteins is selected from Nostoc punctiforme DnaE split, split intein Cfa or split intein Gp41-1.

In an embodiment for better results, the proteolytic activity is a viral protease activity or cellular activity.

In an embodiment for better results, the protease is a virus protease, bacteria protease or cellular protease.

In an embodiment for better results, the output reporter protein is a fluorescent protein or a luminescent protein.

In an embodiment for better results, the virus infection is a tobacco etch virus infection, a adenovirus virus infection, a rhinovirus infection, a ZIKA infection, a chikungunya infection or a coronavirus infection.

Another aspect of the present disclosure relates to a composition comprising the cre pro-enzyme of the present disclosure.

Another aspect of the present disclosure relates to a kit comprising the cre proenzyme of the present disclosure or composition of the present disclosure, for use in detecting viruses and/or proteolytic activity of recombinase.

Another aspect of the present disclosure relates to a virus detection sensor comprising the cre pro-enzyme of the present disclosure.

Another aspect of the present disclosure relates to a method of detecting proteases and quantifing proteolytic activity in a disease or an infection, using the pro-8 enzyme or the composition of the present disclosure, comprising the step of: contacting the pro-enzyme or the compostion with a tissue sample; detecting the reporter protein.

In an embodiment, in the method of the present disclosure, the reporter protein is a fluorescent protein or a luminescent protein; preferably green fluorescent protein.

In an embodiment, in the method of the present disclosure, the infection is a tobacco etch virus infection, a adenovirus virus infection, a rhinovirus infection, a ZIKA infection, a chikungunya infection or a coronavirus infection.

### Amino Acid Abbreviation List

| Amino acid | 3-letter abbreviation | 1-letter abbreviation |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

| | |
|---|---|
| ↓ | clevage point |

### BRIEF DESCRIPTION OF THE DRAWINGS

The following figures provide preferred embodiments for illustrating the disclosure and should not be seen as limiting the scope of invention.

FIG. 1 is a schematic representation of protease-sensing Cre recombinase. Structure models of (A) a cyclized Cre variant and (B) a cleaved Cre sensor variant.

FIG. 2 is a schematic design of the FLEx reporter module.

FIG. 3 shows the results of the validation of the FLEx fluorescent reporter module. *, P<0.05. Scale bar=100 μm.

FIG. 4 shows wwitch-on Cre sensors activated by proteolytic activity. 293T cells were cotransfected with FLEx module, Cre or cyCreTEV, and mock or TEVp coding plasmids. *, P<0.05; ns, not significant; unpaired, two-tailed Student's t-test. Scale bar=100 μm.

FIG. 5 shows switch-on Cre sensors activated by the adenovirus protease. (A) 293T cells were cotransfected with a FLEx module, one of the ProCre variants, and mock or AVP coding plasmids. Western blots for (B) confirmation of ProCre cleavage by adenoviral proteolysis and (C) specificity of the proteolytic cleavage. *, P<0.05; ns, not significant; unpaired, two-tailed Student's t-test. Scale bar=100 μm.

FIG. 6 shows a graphical representation of the chain B: Cre recombinase obtained from the Protein Data Bank (PDB; accession code 1CRX [25]). Sequence as reported in the sample and secondary structure as reported by the Dictionary of Protein Secondary Structure (DSSP).

FIG. 7 shows the characterization of switch-on Cre sensors activated by proteolytic activity.

FIG. 8 shows the results of the evaluation of cysCreTEV with a PEST domain. 293T cells were cotransfected with FLEx module, sensors cysCreTEV or cysCreTEV* (the latter containing a PEST domain), and TEVp or a mock plasmid. *, P<0.05; unpaired, two-tailed Student's t-test.

FIG. 9 shows the characterization of ^{Pro}Cre sensors conditionally activated by adenoviral protease activity.

FIG. 10 shows the specificity of the proteolytic activation of ^{Pro}Cre sensors.

FIG. 11 is a schematic representation of two Cre recombinases complexed with DNA in a site-specific recombination synapse. (A) Two wild type Cre recombinases as retrieved from the PDB (accession code 1CRX). (B) The ^{Pro}Cre (cyclized) is represented as the Cre recombinase in orange. Notice the potential shift in the structure of the C-terminal helix (N), previously buried in a hydrophobic pocket of the adjacent Cre subunit (in yellow), due to the covalent ligation between N- and C-termini by protein cyclization. The obtained model using Modeller software is merely illustrative. Color codes: red, protease cleavable site; cyan, exteins from Npu DnaE split inteins; grey, SV40 NLS; black, DNA strand.

FIG. 12 shows switch-on Cre sensors activated by the rhinovirus protease Evaluation of ^{Pro}Cre^{HRV} for detection of HRV protease activity. Transient co-transfection of 293T cells and analysis of ^{Pro}Cre^{HRV} performance. 293T cells co-transfected with plasmids expressing FLEx, dTEVp or HRVp (−), and either sCre or ^{Pro}Cre^{HRV}, were analysed, 48 h post-transfection, by (A) fluorescence microscopy, and (B) flow cytometry for GFP positive cells (%) and their mean fluorescence intensity (MFI). GFP was quantified in mCherry positive cells (transfected cells). Data shown as mean±SD. Statistical analysis using unpaired, two-tailed Student's t-test. *, P<0.05; ns, nonsignificant. AU, arbitrary units. Scale bar: 100 μm.

### DETAILED DESCRIPTION

The present disclosure relates to compositions and methods for performing conditional homologous recombination of a targeted DNA molecule or genome by using modified proteins comprising nucleic acid DNA binding proteins with protease-conditional recombinase (hereinafter “ProRec”) activity.

In an embodiment, ProRec and FLEx (“flip-excision”) plasmid switch modules were generated. The FLEx cassette coding two nested pairs of incompatible lox sites (loxP and lox2722) and a monomeric (A206K) enhanced green fluorescent protein (GFP) in antisense orientation was designed and synthesized. In the lentiviral transgene cassette pRRLSIN.PGK-GFP PGK promoter and GFP were swapped by the human elongation factor 1 alpha (hEF1α) promoter and the FLEx cassette. Then, mCherry, amplified from pRSET B plasmid, and the Sh ble gene (conferring resistance to Zeocin) preceded by a 77 bp spacer (for reinitiation of translation), amplified from phGaLV10A1, were cloned in sense direction into the FLEx cassette between the loxP and lox2722, originating a double-floxed inverse orientation (DiO) cassette.

In an embodiment, an intermediate construct was used as template for the remaining constructs. For that, in the plasmid of the FLEx module, the DiO cassette was swapped with cVisensor and an encephalomyocarditis virus (EMCV) internal ribosomal entry site (IRES) driving the expression of puromycin resistance gene.

In an embodiment, plasmids identified as Cre, sCre, and TEVp were respectively obtained by replacing cVisensor, in the intermediate construct, with: the full-length Cre recombinase harboring a nuclear localization signal (NLS) from the simian vacuolating virus 40 (SV40), amplified from pZeoCre; a smaller version of Cre recombinase (residues 19-343) harboring a SV40 NLS, amplified from pZeoCre; or a synthesized stable S219V variant of tobacco etch virus protease (TEVp).

In an emdobiment, plasmid cyCreTEV was obtained from the intermediate construct by replacing the circular permuted GFP with the full-length Cre recombinase in fusion with a SV40 NLS and the ENLYFQ↓S cleavable sequence (arrow denoting the scissile bond). In a similar fashion, plasmids identified as cysCreTEV, ^{Pro}Cre-LRGAG, ^{Pro}Cre-EEGEG, and ^{Pro}Cre-EEGEG.v2 were obtained by replacing the circular permuted GFP with a smaller version of Cre recombinase (residues 19-343) in fusion with a SV40NLS and the cleavable sequence ENLYFQ↓LS, LRGA↓LG, EEGE↓G, or LEEGE↓GLARL, respectively. The plasmid identified as cysCreTEV* was obtained from plasmid cysCreTEV by replacing the myc-tag with the PEST degradation domain from mouse ornithine decarboxylase.

In an emdobiment, a plasmid simulating a cleaved ^{Pro}Cre-LRGAG, named linear ^{Pro}Cre, was obtained by replacing cVisensor with a ^{Pro}Cre-LRGAG without flanking Npu DnaE split inteins. A mock plasmid was obtained by replacing GFP in the original pRRLSIN.PGK-GFP by lacZ gene (amplified from pIRESGALEO). The construction of the plasmid coding for the AVP. All constructs were generated using standard molecular biology techniques and confirmed by exhaustively sequencing the cloned fragments.

In an embodiment, evaluation and application of ProRec and FLEx modules were performed using mammalian cell lines. 293T cell line was routinely cultivated in Dulbecco's modified Eagle's medium, supplemented with 10% (v/v) of fetal bovine serum (FBS), and maintained at 37° C. in an incubator with humidified atmosphere of 5% CO₂.

In an embodiment, FLEx reporter and Cre sensor modules were screened.

In an embodiment, development and characterization of the FLEx module and the different variants of Cre sensor module were performed by transient transfection. 293T cells seeded at 8×10⁴ cells/cm² the day before were cotransfected with 5 μg of total DNA/(10⁶ cells) using linear 25 kDa poly (ethylenimine) (PEI). After 48 hours, cells were (i) observed by fluorescence microscopy; (ii) assessed by flow cytometry (gates set using non-transfected 293T as negative control and the percentage and geometric mean GFP fluorescence intensity (MFI) of cells within the positive gate were measured); or (iii) analyzed by Western blotting.

In an embodiment, Western blotting was performed. 293T cells transiently transfected were lysed with Protein Extraction Reagents supplemented with a EDTA-free Protease Inhibitor Cocktail. Equal amounts of protein (30 ug) were loaded and resolved in 12% (w/v) SDS-PAGE gels and then transferred to nitrocellulose membranes. Anti Cre recombinase anti myc-tag, anti α-tubulin, and HRP-conjugated sheep anti-mouse IgG or donkey anti-rabbit were used as antibodies.

Results are shown as mean±standard deviation (SD) of three independent experiments (unless otherwise stated). Statistical significance was evaluated by comparing samples to their respective mock controls using one-way analysis of variance (ANOVA) followed by Dunnett's posthoc test, or unpaired, two-tailed Student's t-test. P values<0.05 were considered statistically significant.

In an embodiment, switch-on Cre recombinase sensor and flexible reporter modules were designed.

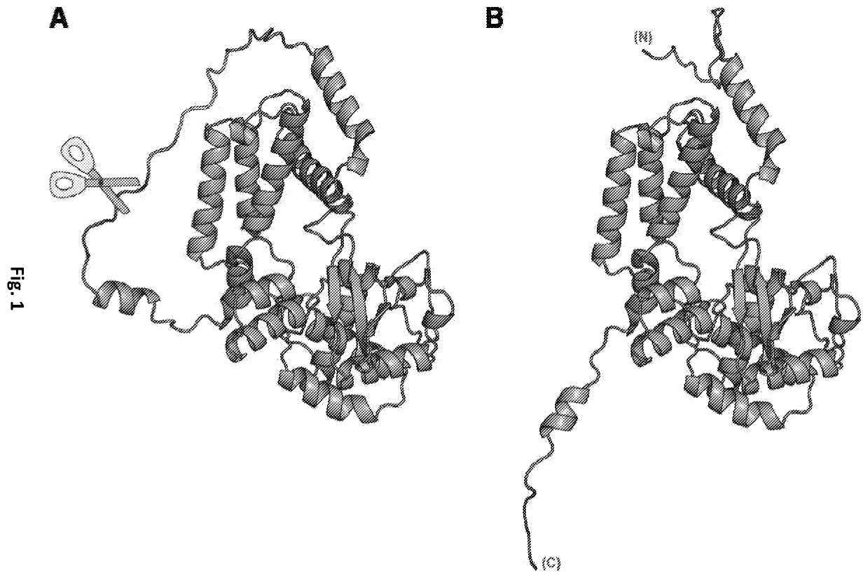

In an embodiment, the genetically encoded Cre recombinase variant of the present disclsoure, when expressed, remains in a non-functional pro-enzyme state. As such, cyclization of Cre promoted by the efficient Npu DnaE split intein renders an enzyme impaired due to the imposed structural constraints (FIG. 1A). The impaired activity of the cyclized Cre variant would be conditionally reconstituted upon proteolysis of a protease-specific cleavable sequence (FIG. 1).

FIG. 1 shows is a schematic representation of protease-sensing Cre recombinase. Structure models of (FIG. 1A) a cyclized Cre variant and (FIG. 1B) a cleaved Cre sensor variant. Illustrative models were obtained using Protein Data Bank accession code 1CRX as template and performing comparative modeling using Modeller software. Color codes: red, protease cleavable site; cyan, exteins from Npu DnaE split inteins; grey, SV40 NLS. Scissor representing viral protease. Illustrations not at scale.

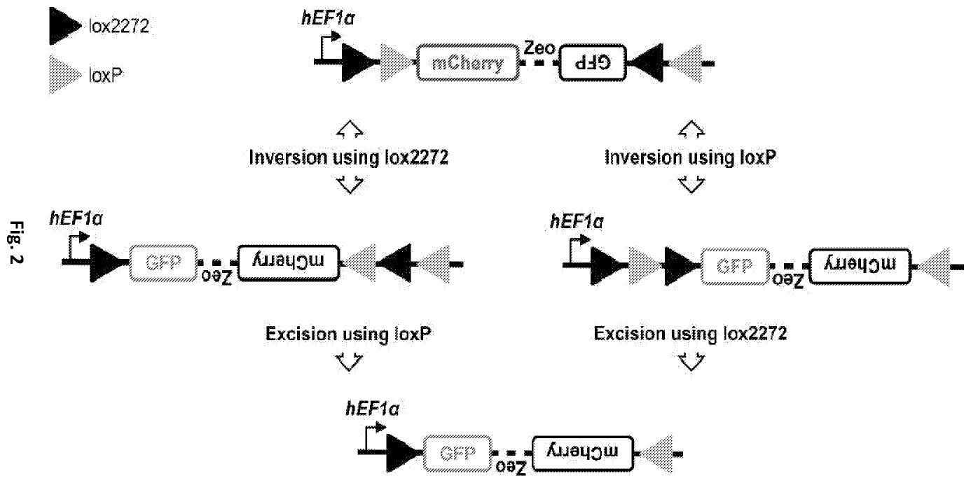

In an embodiment, to allow a potential transient proteolytic event to be transformed into a permanent and cell heritable amplified signal, a modular switch-on reporter was created. By taking advantage of the FLEx system and of pairs of the heterotypic loxP and lox2272 sites, a double-floxed inverse orientation (DiO) cassette (FIG. 2) was designed. mCherry is constitutively expressed until a protease-activated Cre performs a flip-excision event. The DNA fragment containing mCherry-Zeo and GFP (the latter in antisense orientation) are first reversibly flipped (to their reverse complement) via either pair of lox sites, enabling a second and irreversible excision event (preventing further inversion) with constitutive expression of GFP expression.

FIG. 2 shows a schematic design of the FLEx reporter module. FLEx system makes use of two pairs of heterotypic—loxP and lox2272—and antiparallel sites. Due to the specific positioning and orientation of the lox pairs, an inversion event-via either loxP or lox2272 sites—followed by an excision event—leading to excision of two lox sites—turns off mCherry while constitutively turning on GFP expression.

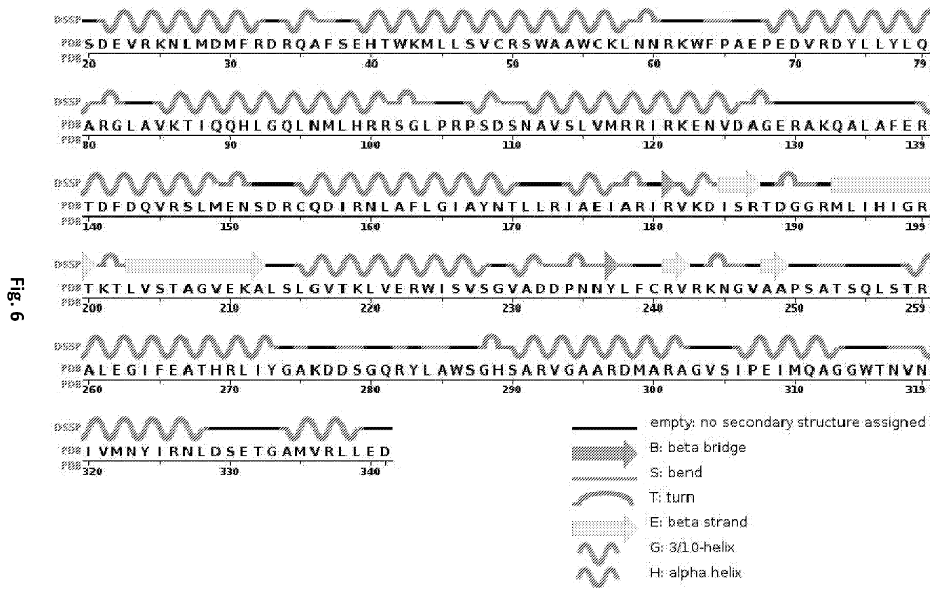

In an embodiment, the FLEx reporter module was designed and tested through expression of Cre recombinase. Analysis of the X-ray crystal structure (FIG. 6) shows that Cre enzyme folds into two distinct domains: the amino-terminal domain (residues 20-129), and the carboxy-terminal domain (residues 132-341). Due to the unstructured nature of the initial amino terminal residues, in addition to full-length Cre variant (Cre), a truncated Cre variant (residues 19-343; ₛCre) was also developed and evaluted. The functionality of the FLEx module was assessed by co-transfection of 293T cells with Cre or sCre.

FIG. 6 shows graphical representation of the chain B: Cre recombinase obtained from the Protein Data Bank (PDB; accession code 1CRX). Sequence as reported in the sample and secondary structure as reported by the Dictionary of Protein Secondary Structure (DSSP).

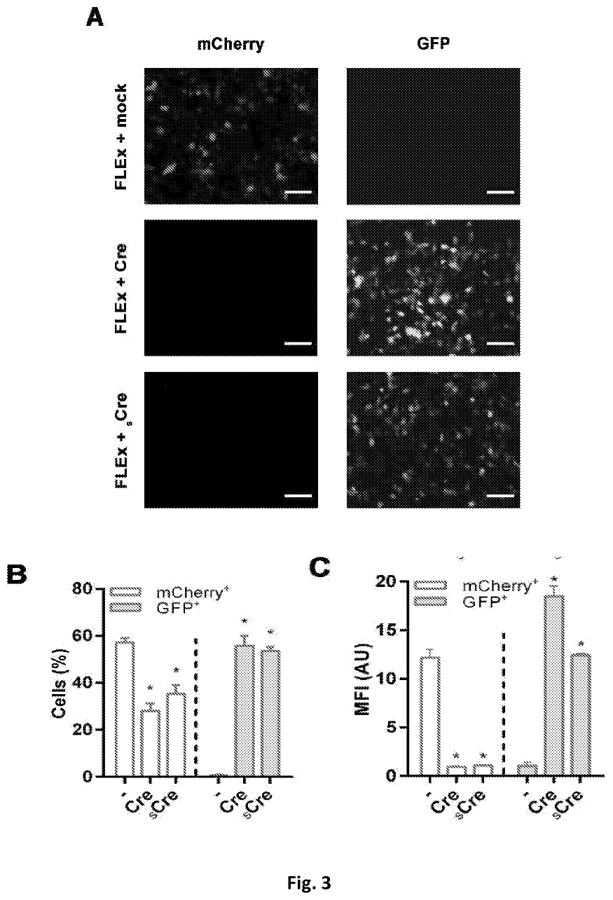

In the absence of recombinase, only mCherry fluorescence was detected (FIG. 3A). Upon addition of Cre, mCherry fluorescence decreased significantly while switching-on a robust GFP expression (FIGS. 3B and C). When compared to Cre, truncated variant ₛCre yielded a similar percentage of GFP⁺ cells, although with less mean fluorescence intensity (MFI; FIG. 3C). These results show that the design of the FLEx module, which provides a high performing dark-to-bright reporter, was successful.

FIG. 3 shows validation of the FLEx fluorescent reporter module. To evaluate the FLEx reporter module, 293T cells were cotransfected with FLEx reporter and a mock, Cre, or sCre coding plasmids. After 48 h, cells were (A) visualized by fluorescence microscopy and (B and C) analyzed by flow cytometry for percentage of cells expressing mCherry and GFP and its mean fluorescence intensity (MFI), respectively. Statistical analysis was performed by comparing Cre and sCre to the negative control (mock transfected) with an ANOVA followed by Dunnett's posthoc test. *, P<0.05. Scale bar=100 μm.

In an embodiment, proteolysis activation of switch-on Cre recombinase sensors were determined.

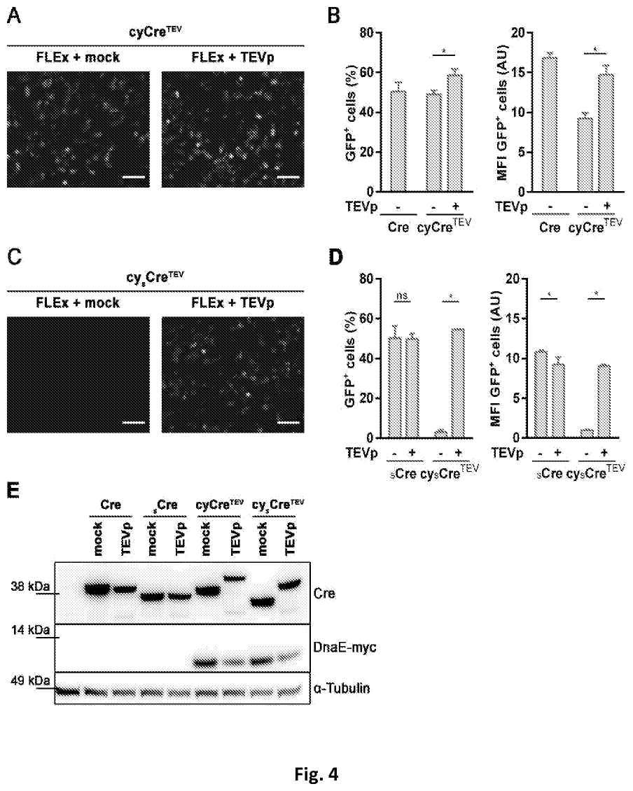

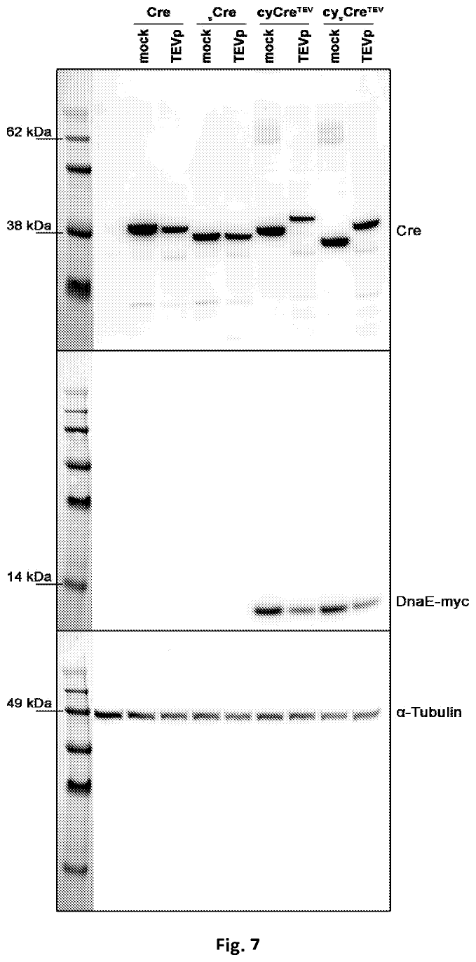

To tightly control the always-on nature of the Cre recombinase, cyclized Cre variants were designed and characterized. In parallel, to assess the applicability of these variants as switch-on sensors activated by proteolytic activity, both Cre and ₛCre were fused to a 7 residue cleavable sequence (ENLYFQ↓S) specifically and efficiently recognized by the tobacco etch virus protease (TEVp) [28]. As shown in FIG. 4.A, cyclization of full-length Cre in fusion with a TEVp cleavable sequence (cyCre^{TEV}) did not impair its functionality, as GFP fluorescence was well detected even before TEVp activity. Flow cytometry analysis further validated these observations. Despite no appreciable increase in percentage of GFP⁺ cells, analysis of GFP⁺ cells MFI showed an increase in 60% upon proteolysis of the cyCre^{TEV} variant (FIG. 4B). Conversely, cyclization of the truncated ₛCre (cyₛCre^{TEV}) significantly impaired its function (FIG. 4C) as only a small fraction of cells (3.2%) emitting low GFP fluorescence was detected by flow cytometry (FIG. 4D). It was evaluated to determine if the resulting switch-on of GFP fluorescence was indeed due to proteolysis-activation of the cyclized variants. For that, protein extracts of cotransfected 293T cells were analyzed by Western blot (FIG. 4E). Full cyclization of cyCre^{TEV} and cyₛCre^{TEV} was observed by the appearance of the 13 kDa myc-tagged N-fragments of the Npu DnaE split intein, and absence of myc-tagged non-cyclized Cre (59 kDa and 57 kDa, theoretical molecular weights, respectively; FIG. 7).

FIG. 4 shows switch-on Cre sensors activated by proteolytic activity. 293T cells were cotransfected with FLEx module, Cre or cyCreTEV, and mock or TEVp coding plasmids. After 48 h, cells were (FIG. 4A) visualized by fluorescence microscopy and (FIG. 4B) analyzed by flow cytometry. 293T cells were cotransfected with FLEx module, sCre or cysCreTEV, and mock or TEVp coding plasmids. After 48 hours, cells were (FIG. 4C) visualized by fluorescence microscopy and (FIG. 4D) analyzed by flow cytometry. (FIG. 4E) 293T cells were transiently cotransfected with the indicated plasmids. After 48 hours, cell extracts were generated, resolved in 12% (w/v) SDS-PAGE gels, and analyzed by Western blotting. Cre variants were detected with anti Cre primary antibody. Cyclization was detected (with anti myc-tag primary antibody) by release of myc-tagged N-fragment of Npu DnaE intein (DnaEn-myc). Molecular weights indicated in the figure correspond to proteins of SeeBlue Plus2 Pre-Stained Protein Standard (Invitrogen). *, P<0.05; ns, not significant; unpaired, two-tailed Student's t-test. Scale bar=100 μm.

FIG. 7 shows characterization of switch-on Cre sensors activated by proteolytic activity. Full-length western blots of the cropped version presented in FIG. 4E. Cre variants were detected with anti Cre primary antibody. Cyclization was detected (with anti myc-tag primary antibody) by release of myc-tagged N-fragment of Npu DnaE intein (DnaEn-myc).

In an embodiment, cleavage of the cyclized variants was confirmed only in presence of TEVp, as seen by the appearance of proteins with 41 kDa and 40 kDa (theoretical molecular weights), respectively for cyCre^{TEV} and cyₛCre^{TEV}, running slower in the gel than the cyclized (not cleaved) counterparts. Proteins with>62 kDa was detected only with anti Cre recombinase antibody but not with anti myc-tag antibody; this may correspond to cyclized bimolecular sensors (FIG. 7).

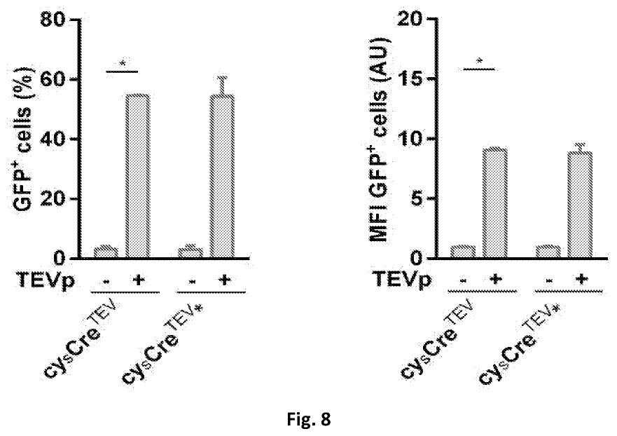

In an embodiment, to further confirm the full cyclization of the cyₛCre^{TEV} variant, as any non-cyclized enzyme remains highly active, the myc-tag was replaced with a PEST degradation domain from mouse ornithine decarboxylase. Flow cytometry analysis showed that no further reduction of the low background activity of the cyₛCre^{TEV} variant (FIG. 8) was observed.

FIG. 8 shows evaluation of cysCreTEV with a PEST domain. 293T cells were cotransfected with FLEx module, sensors cysCreTEV or cysCreTEV* (the latter containing a PEST domain), and TEVp or a mock plasmid. After 48 h, cells were analyzed by flow cytometry for percentage of GFP+ cells and their mean fluorescence intensity (MFI). Error bars of cysCreTEV* samples result from two independent experiments. *, P<0.05; unpaired, two-tailed Student's t-test.

These results show that cyclization of the truncated ₛCre, but not of full-length Cre, tightly controls the activity of the recombinase, and that cyₛCre (hereafter referred as ^{Pro}Cre, protease-sensing Cre recombinase) can be used as a sensor conditionally activated by proteolytic activity.

In an embodiment, the ability of the ProRec (^{Pro}Cre) protein of the present disclosure in sensing adenoviral proteolytic activity was evaluated.

In an embodiment, it was evaluated if the design of switch-on Cre recombinase could be generalized to other proteases. Human adenoviruses are responsible for multiple diseases, from epidemic keratoconjunctivitis to more severe multiple-organ failure. When engineered, adenoviruses are also widely used in clinical applications such as oncolytic virotherapy. Adenoviruses code a well-studied protease—adenovirus protease, AVP—belonging to a different family of the TEVp. As such, the cleavable sequence on ^{Pro}Cre was changed to LRGA↓G, EEGE↓G, and a larger LEEGE↓GLARL (the latter referred as EEGEG.v2 for simplicity), recognized by the AVP.

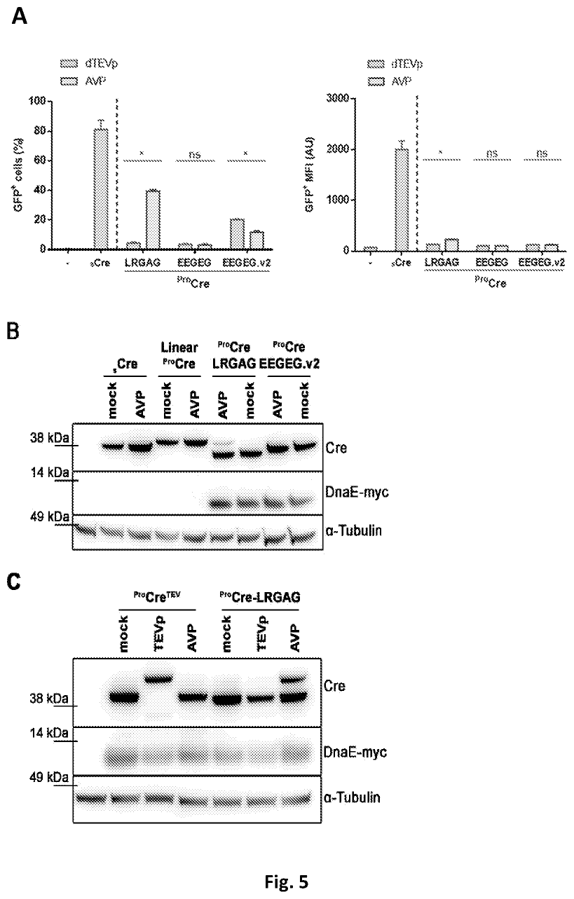

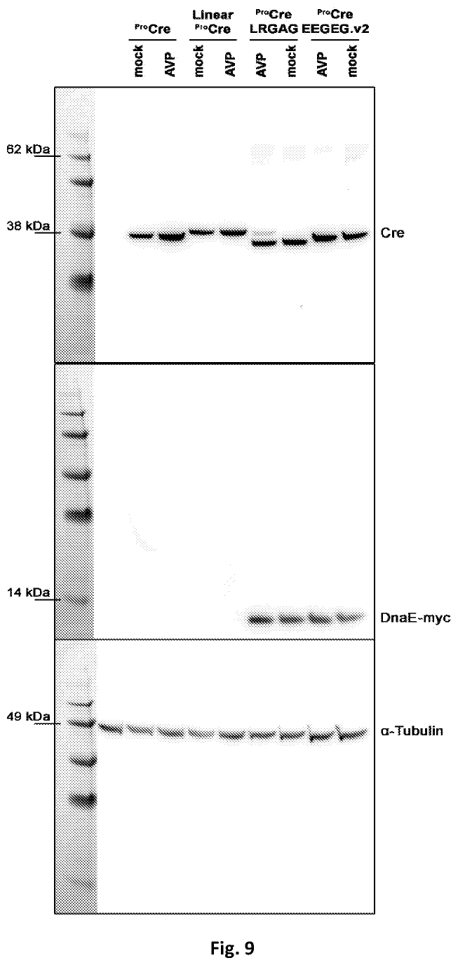

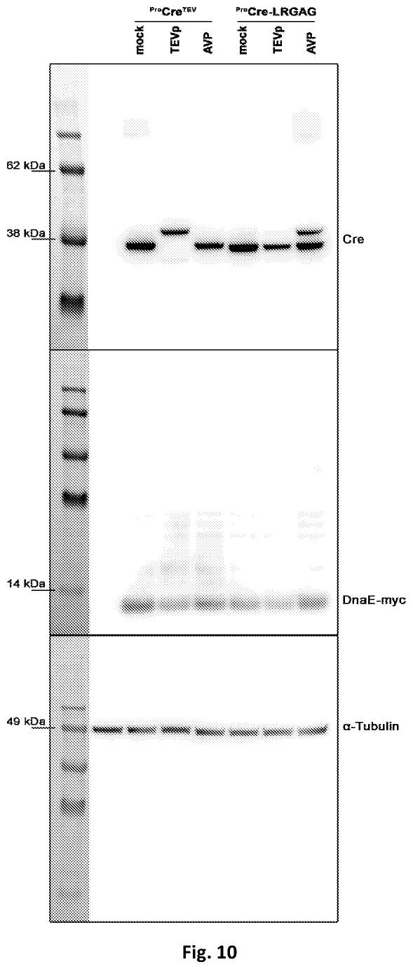

Screenings performed by cotransfection of 293T cells with FLEx module, one of the ^{Pro}Cre variants, and AVP showed increase in both percentage of GFP+ cells or fluorescence intensity (FIG. 5A). Modification of the FLEx module, where mCherry was removed allowed to increase GFP fluorescence emission in the presence of AVP. Analysis by flow cytometry revealed that the percentage of GFP positive cells increased from 4.5±0.6% to 40±1% (FIG. 5A), followed by a slight increase of 1.7-fold in fluorescence intensity (FIG. 5A). This was observed for LRGAG cleavage sequences. Western blot analysis showed cleavage of ^{Pro}Cre-LRGAG variant (albeit only a fraction of the cyclized form), but not of the ^{Pro}Cre-EEGEG.v2 variant (FIG. 5B and FIG. 9). Additionally ^{Pro}Cre variants were only cleaved by the cognate proteases, suggesting that cleavage is indeed specific and taking place at the cleavable sequence (FIG. 10C and FIG. 10).

FIG. 5 shows switch-on Cre sensors activated by the adenovirus protease. (A) 293T cells were cotransfected with a FLEx module, one of the ^{Pro}Cre variants, and mock or AVP coding plasmids. After 48 h, cells were analyzed by flow cytometry for percentage of GFP+cells and their mean fluorescence intensity (MFI). Western blots for (B) confirmation of ^{Pro}Cre cleavage by adenoviral proteolysis and (C) specificity of the proteolytic cleavage. Briefly, 293T cells were cotransfected with the indicated plasmids. After 48 h, cell extracts were generated, resolved in 12% (w/v) SDS-PAGE gels. Cre variants were detected with anti Cre primary antibody. Cyclization was detected (with anti myc-tag primary antibody) by release of myc-tagged N-fragment of Npu DnaE intein (DnaEn-myc). Molecular weights are indicated in the figure. Error bars of ^{Pro}Cre-LRGAG samples result from two independent experiments. *, P<0.05; ns, not significant; unpaired, two-tailed Student's t-test. Scale bar=100 μm.

FIG. 9 shows characterization of ^{Pro}Cre sensors conditionally activated by adenoviral protease activity. Full-length western blots of the cropped version presented in FIG. 5B. Cre variants were detected with anti Cre primary antibody. Cyclization was detected (with anti myc-tag primary antibody) by release of myc-tagged N-fragment of Npu DnaE intein (DnaEn-myc).

FIG. 10 shows specificity of the proteolytic activation of ^{Pro}Cre sensors. Full-length western blots of the cropped version presented in FIG. 5C. Cre variants were detected with anti Cre primary antibody. Cyclization was detected (with anti myc-tag primary antibody) by release of myc-tagged N-fragment of Npu DnaE intein (DnaEn-myc).

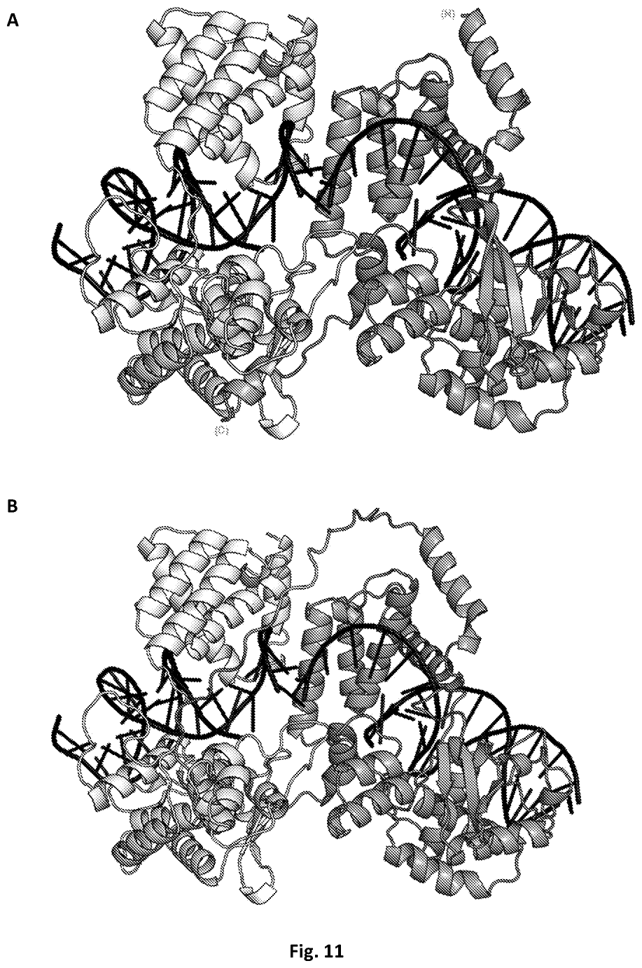

In an embodiment, the activity of Cre recombinase was made controllable by fusing the N-and C-termini of Cre recombinase to split inteins, structural distortion induced upon cyclization of Cre chimeras effectively promotes a nonfunctional pro-enzyme state. The first approach comprises cyclization of full-length Cre; however, its activity was not completely impaired (FIGS. 4A and 4B). As the N-and C-termini of Cre are thought to be located in close proximity, cyclization did not significantly perturbed its native structure. In contrast, cyclization of a truncated version of Cre (sCre, residues 19-343) allowed its inactivation, as only a small fraction of cells (3.2%) emitting low GFP fluorescence was detected (FIGS. 4C and 4D). This may nevertheless translate to even lower (or complete absence of) background activity in stable conditions, as transient screenings are performed in high protein expression conditions. These results suggest that major perturbations in the structure of the sCre variants are induced upon protein cyclization. Indeed, both the first helix of the amino-terminal domain Cre (helix A, residues 21-32) as well as the last helix of the carboxy-terminal domain (helix N, residues 334-339) are involved in formation of the recombinase tetramer and intersubunit contacts. Moreover, the tyrosine nucleophile (Y324) locates close to the peptide linker (R326-G333) that connects helix N to the rest of the domain. As such, covalent peptide ligation of the N-and C-termini of the truncated sCre may affect the tertiary structure of these important helices and, consequently, impair the quaternary structure of the recombination complex (FIG. 11).

FIG. 11 shows schematic representation of two Cre recombinases complexed with DNA in a site-specific recombination synapse. (FIG. 11A) Two wild type Cre recombinases as retrieved from the PDB (accession code 1CRX). (FIG. 11B) The ProCre (cyclized) is represented as the Cre recombinase in orange. Notice the potential shift in the structure of the C-terminal helix (N), previously buried in a hydrophobic pocket of the adjacent Cre subunit (in yellow), due to the covalent ligation between N- and C-termini by protein cyclization. The obtained model using Modeller software is merely illustrative. Color codes: red, protease cleavable site; cyan, exteins from Npu DnaE split inteins; grey, SV40 NLS; black, DNA strand.

Covalent linkage of N-and C-termini has been exploited to create circular permuted zymogen pro-enzymes. However, when Cre recombinase protein sequence is split it often leads to chimeras with lower DNA recombination efficiency-as low as 23% when compared to the full-length version. Herein, the truncated sCre maintains 77% of its DNA recombination efficiency when compared to full-length Cre, as detected by the lower GFP MFI that can be correlated to number of inverted copies of the FLEx module (FIGS. 3C). This contrasts with a previous report indicating that truncated Cre retained similar recombinase activity as full-length Cre. As activity of cyclized sCre variants (cysCre) is tightly controlled, it was envisioned that if the linker sequence fusing the N- and C-termini contained a cleavable sequence recognized by proteases, a switch-on Cre recombinase could be designed. As proof-of-concept, cysCre fused to a 7 residue cleavable sequence recognized by the TEVp (cysCreTEV) was shown to activate a robust GFP signal (FIGS. 4C and 4D) upon TEVp specific proteolysis (FIG. 4E). Importantly, recombination efficiency of this chimera is similar to non-cyclized sCre, as GFP MFI levels are identical (FIG. 4D).

Overall, these results showed that Cre recombinase could be rewired into a protease-sensing Cre recombinase (^{Pro}Cre). As such, applicability of this innovative design was assessed for detection of proteolytic activity of the clinically and biotechnologically important human adenovirus. GFP signal was detected upon addition 20 of AVP (FIG. 5A). Detection of specific proteolytic cleavage of the cyclized chimeras was also observed (FIGS. 5B and 5C). The activity is lower than with TEVp protease. Two hypothesis can be suggested. Contrasting remarkably to the total cleavage of cysCreTEV by TEVp (FIG. 4E), the low ratio of cleaved to cyclized ^{Pro}Cre-LRGAG-explained by partially activated AVP, or steric hindrance blocking AVP from reaching the cleavable sequence—might perturb DNA recombination event in the FLEx module. Further characterization and optimizations may lead to more efficient ^{Pro}Cre detection of AVP activity.

In an embodiment, a switch-on Cre recombinase chimera was created by means of protein cyclization. ProRec remains in a tightly controlled off state, until specific proteolytic activation. Those transient events are then transformed into permanent and robust fluorescent signals easily detectable. Moreover, the modularity of ProRec system enables simple adaptation to other proteases—viral or cellular, by exchanging the cleavable sequence—and output signals—by exchanging the reporter gene in the FLEx module. As genetically encoded elements, both modules can be applied in the development of cellular platforms reporting cell-specific pathways, virus detection and quantification, or drug screening.

TABLE 1
Elements on the sequences of the developed constructs.

| | | | |
|---|---|---|---|
| Name | Promoter | Construct | Selection |

FLEx module

| | | | |
|---|---|---|---|
| FLEx (DiO) | hEF1α | lox>-mCherry-Zeo-meGFP-<lox | — |

Cre variants

| | | | |
|---|---|---|---|
| Cre | hEF1α | nls-Cre | IRES-Puro |
| ₛCre | hEF1α | nls-ₛCre | IRES-Puro |

TEV protease

| | | | |
|---|---|---|---|
| TEVp | hEF1α | TEVp | IRES-Puro |

Cyclized Cre sensor modules for TEV protease

| | | | |
|---|---|---|---|
| cyCre^{TEV} | hEF1α | Dc-EF-nls-Cre-ENLYFQ↓S-GS- | IRES-Puro |
| CyₛCre^{TEV} | hEF1α | Dc-EF-nls-ₛCre-ENLYFQ↓S-GS- | IRES-Puro |
| CyₛCre^{TEV}* | hEF1α | Dc-EF-nls-ₛCre-ENLYFQ↓S-GS- | IRES-Puro |

ProCre sensor modules for AVP

| | | | |
|---|---|---|---|
| ^{Pro}Cre-LRGAG | hEF1α | Dc-EF-nls-ₛCre-LRGA↓G-GS-Dn- | IRES-Puro |
| ^{Pro}Cre-EEGEG | hEF1α | Dc-EF-nls-ₛCre-EEGE↓G-GS-Dn- | IRES-Puro |
| ^{Pro}Cre-EEGEG.v2 | hEF1α | Dc-EF-nls-ₛCre-LEEGE↓GLARL- | IRES-Puro |
| Linear ^{Pro}Cre | hEF1α | G-GS-AEY-CFN-EF-nls-ₛCre-LRGA | IRES-Puro |

Cleavable sequences are represented in bold, with arrow denoting the scissile bond. Lox>, lox2272>and loxP>sites, separated by a 50 bp spacer; mCherry, mCherry fluorescent protein; Zeo, Sh ble gene linked to mCherry by a 77 bp spacer (for reinitiation of translation), conferring resistance to Zeocin; meGFP, monomeric (A206K) enhanced GFP in antisense orientation; <lox, <lox2272 and <loxP sites, separated by a 50 bp spacer; Dc, C-fragment of Npu DnaE split intein and CFN residues of C-extein; EF, residues coded by EcoRI endonuclease restriction site; nls, SV40 NLS; Cre, full-length Cre recombinase; ₛCre, residues 19 to 343 of full-length Cre; GS, residues coded by BamHI endonuclease restriction site; Dn, AEY residues of N-extein and N-fragment of Npu DnaE split intein; myc, epitope tag derived from c-Myc protein with a GGGGS flexible linker; PEST, degradation domain from mouse ornithine decarboxylase with a GGGGS flexible linker; IRES, encephalomyocarditis virus internal ribosomal entry site; Puro, pac gene for resistance to puromycin; TEVp, S219V variant of tobacco etch virus protease.

In an embodiment, the 3C protease of the clinically relevant human rhinovirus 14B, herein HRVp, was also chosen for exemplifying the the ProRec use. Human rhinovirus (HRV) is the most common human respiratory pathogen, responsible for the majority of common colds, and has a very well-studied protease with unique specificity.

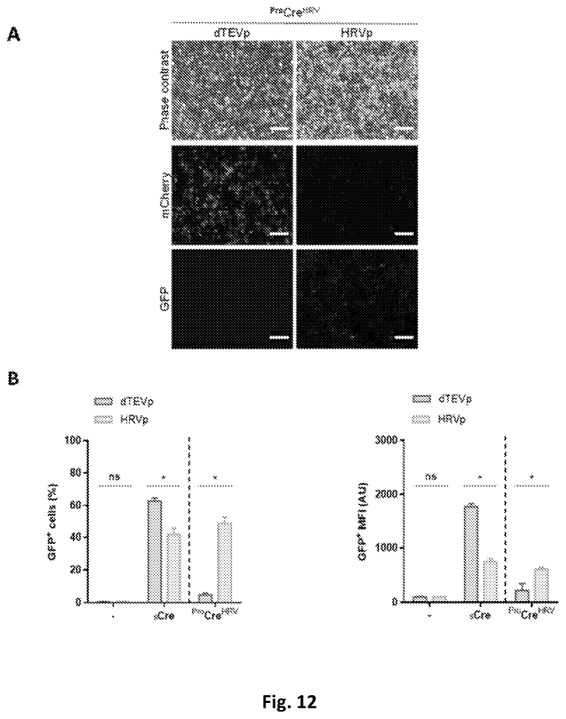

In an embodiment, for sensor validation with HRVp, cyₛCre^{TEV} cleavable sequence was replaced with LEVLFQ↓GP (arrow denotates HRVp scissile bond), originaOng ^{Pro}Cre^{HRV}. Cells were then transiently transfected with FLEx.2, sCre or ^{Pro}Cre^{HRV}, and either dTEVp (used here as negative control) or HRVp (FIG. 12).

Phase-contrast images of cells transfected with HRVp showed an increased cell death when compared with cells transfected with dTEVp (FIG. 12A). Nonetheless, GFP fluorescence emission increased in presence of HRVp, indicating a protease-dependent activation.

^{Pro}Cre^{HRV} lead to an increase from 5±1% to 49±4% GFP+ cells (FIG. 12B), and an almost 3-fold increase in MFI of GFP+ cells, with HRVp (FIG. 12B). HRVp, ProCre HRV and sCre percentage of GFP+ cells (49±4% and 42±4%, respectively) and GFP+ MFI (752±71 AU and 610±38 AU, respectively) was very similar.

In an embodiment, Western blotting analysis was performed to verify if the increase in GFP fluorescence emission in the presence of HRV protease was due to proteolytic cleavage. Results confirm specific cleavage. Overall, these results revealed that ProCreHRV can detect proteolytic activity efficiently thus, the system can also be used to detect human rhinovirus.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

1. A Cre recombinase pro-enzyme, for identifying and quantifying proteolytic activity in a disease or an infection, wherein said cre recombinase pro-enzyme comprising intein mediated circularization, a flip-excision cassette comprising an output effector and/or reporter protein, and a linker sequence cleavable by a protease;
wherein the intein mediated circularization is a N-terminal-C-terminal fused split intein mediated circularization; and
wherein the cleavable linker sequence comprises from 4-15 amino acids.

2. The pro-enzyme of claim 1, wherein the flip-excision cassette comprises the reporter protein.

3. The pro-enzyme of claim 1, wherein the flip-excision cassette comprises the output effector protein.

4. (canceled)

5. (canceled)

6. The pro-enzyme of claim 1, wherein the linker sequence cleavable is selected from
SEQ. ID. 1: ENLYFQ↓S, SEQ. ID. 2: LRGA↓G, SEQ. ID. 3: EEGE↓G, SEQ. ID. 4: LEEGE↓GLARL, SEQ. ID. 5: LRGA↓G, SEQ. ID. 6: EEGE↓G, SEQ. ID. 7: LEVLFQ↓GP, SEQ. ID. 8: RAGG↓YIFS, SEQ. ID. 9: DELRLDRAGG↓YIFSS, SEQ. ID. 10: RAGA↓GIIE, SEQ. ID. 11: VEQLEDRAGA↓GIIET, SEQ. ID. 12: ERKRR↓GAD, SEQ. ID. 13: AAGKR↓GAA, SEQ. ID. 14: LVKRR↓GGG, SEQ. ID. 15: SAVLQ↓SGF, 24 SEQ. ID. 16: VARLQ↓SGF, SEQ. ID. 17: VVRLQ↓SGF.

7. The pro-enzyme of claim 1, wherein the split inteins is selected from: Nostoc punctiforme DnaE split, split intein Cfa, or split intein Gp41-1.

8. The pro-enzyme of claim 1, wherein the proteolytic activity is a virus activity or cellular activity.

9. The pro-enzyme of claim 1, wherein the protease is a virus protease, bacteria protease or cellular protease.

10. The pro-enzyme of claim 1, wherein the reporter protein is a fluorescent protein or a luminescent protein.

11. The pro-enzyme of claim 1, wherein the infection is a tobacco etch virus infection, a adenovirus virus infection, a rhinovirus infection, a ZIKA infection, a chikungunya infection or a coronavirus infection.

12. A composition comprising the pro-enzyme of claim 1.

13. A kit comprising the pro-enzyme of claim 1 for use in detecting viruses and/or proteolytic activity of recombinase.

14. A virus detection sensor comprising the pro-enzyme of claim 1.

15. A method of detecting proteases and quantifying proteolytic activity in a disease or an infection, using the pro-enzyme of claim 1, comprising the steps of: contacting the pro-enzyme with a tissue sample; and detecting the reporter protein.

16. The method of claim 15, wherein the reporter protein is a fluorescent protein or a luminescent protein.

17. The method of claim 15, wherein the infection is a tobacco etch virus infection, a adenovirus virus infection, a rhinovirus infection, a ZIKA infection, a chikungunya infection or a coronavirus infection.

18. The pro-enzyme of claim 1, wherein the cleavable linker sequence comprises from 5-12 amino acids.

19. The pro-enzyme of claim 1, wherein the cleavable linker sequence comprises from 6-10 amino acids.
